(19) United States
(12) Patent Application Publication
(10) Pub. No.: US 2022/0365063 A1
(43) Pub. Date: Nov. 17, 2022

(54) GROUP B STREPTOCOCCUS INFECTION

(71) Applicant: THE UNIVERSITY OF WARWICK, Coventry (GB)

(72) Inventors: James COVINGTON, Coventry (GB); Lauren LACEY, Coventry (GB); Siobhan QUENBY, Coventry (GB)

(21) Appl. No.: 17/762,710

(22) PCT Filed: Sep. 24, 2020
(86) PCT No.: PCT/GB2020/052318

§ 371 Date: Mar. 22, 2022

(30) Foreign Application Priority Data
Sep. 25, 2019 (GB) 1913798.3

(51) Int. Cl. G01N 33/487

(52) U.S. Cl. CPC G01N 33/487; G01N 2030/884

(57) ABSTRACT

A screening method for confirming that a subject does not have a Group B Streptococcus (GBS) infection, the method comprising: determining if a GBS-volatile organic compound (VOCs) is not present in a sample that has been taken from the genital mucosa of the subject, wherein if a GBS-VOC is not present in the sample, the subject does not have a Group B Streptococcus infection. A method of diagnosing that a subject has a Group B Streptococcus (GBS) infection, the method comprising: determining if a GBS-volatile organic compound (VOCs) is present in a sample that has been taken from the genital mucosa of the subject, wherein if a GBS-VOC is present in the sample, the subject has a Group B Streptococcus infection.

### FIELD OF THE INVENTION

The present invention relates to a screening method for confirming that a subject does not have a Group B Streptococcus (GBS) infection and a method of diagnosing a subject that does have a GBS infection. The invention also includes a method of treating a subject that has a Group B Streptococcus infection, and extends to a method of determining if a therapeutic agent is effectively treating a Group B Streptococcus infection in a subject. The invention also relates to use of a GBS volatile organic compound (VOC) as a biomarker for determining if a subject has a GBS infection.

### BACKGROUND

Streptococcus agalactiae or Group B Streptococcus (GBS) is the most frequent cause of life threatening early onset infection in new born infants in the UK, known as EOGBS disease. The incidence of EOGBS disease in the UK and Ireland in 2015 was 0.57/1000 births, which is a significant increase in incidence since the previous surveillance was undertaken in 2000 (0.48/1000). GBS infection can lead to sepsis, pneumonia, meningitis and death.

GBS commonly colonises the gastrointestinal and genital tract of adults, with a global mean prevalence of 17.9%. It only rarely causes disease in the immunocompromised adult but it can pose a significant risk to the health of newborn infants due to their immature immune systems. The infection is acquired vertically though exposure to GBS from the vagina of a colonised mother.

The optimal screening strategy to prevent EOGBS is uncertain, with a variety of practices worldwide. Maternal colonisation with GBS is the primary risk factor for disease (transmission to newborns is 40 to 70% and of these 1 to 2% will develop an infection). Guidelines advocate that intrapartum antibiotics should be offered to women found to be colonised during pregnancy and to women with other risk factors, as this has been demonstrated to reduce the risk of culture positive EOGBS disease in the neonate.

However, GBS colonisation status is often intermittent and can be transient during pregnancy. Up to 13% of women who are GBS positive in mid-trimester receive unnecessary prophylactic antibiotics during labour, which may contribute to the increasing prevalence of antibiotic resistance bacteria. The universal screening policy in the US tests women between 35 to 37 weeks of pregnancy. Studies have found that among women who receive a negative GBS screening result, 2 to 10% will become colonised before the onset of labour. Consequently, in the US, some women undergo labour while being colonised by undetectable levels of GBS.

The currently used diagnostic methods for colonisation with GBS utilise time-consuming enrichment culture methods and therefore are not appropriate for an intrapartum scenario.

There is therefore a need for an improved method of diagnosing maternal colonisation with Group B Streptococcus (GBS).

### STATEMENTS OF THE INVENTION

According to a first aspect of the invention, there is provided a screening method for confirming that a subject does not have a Group B Streptococcus (GBS) infection, the method comprising:
determining if a GBS-volatile organic compound (VOCs) is not present in a sample that has been taken from the genital mucosa of the subject,
wherein if a GBS-VOC is not present in the sample, the subject may not have a Group B Streptococcus infection.

However, if a GBS-VOC is present in the sample, the subject may have a GBS infection.

According to second aspect of the invention, there is provided a method of diagnosing that a subject has a Group B Streptococcus (GBS) infection, the method comprising:
determining if a GBS-volatile organic compound (VOCs) is present in a sample that has been taken from the genital mucosa of the subject,
wherein if a GBS-VOC is present in the sample, the subject may have a Group B Streptococcus infection.

The method according to the invention may further comprise a step of confirming that the subject does not have a GBS infection if a GBS-VOC is not present. The method according to the invention may further comprise a step of confirming that the subject does have a GBS infection if a GBS-VOC is present or diagnosing a subject that has a GBS infection if a GBS-VOC is present.

Volatile organic compounds may be defined as organic compounds that have a low boiling point at atmospheric pressure. Consequently, they form a liquid phase, which evaporates/diffuses also forms a gaseous phase.

The inventors have surprisingly found that subjects infected with GBS produce a unique VOC fingerprint (i.e. GBS-VOCs). Advantageously, therefore, the invention can be used to reliably determine if a subject has a GBS infection and distinguish a GBS infection from other bacterial infections and/or fungal infections. Moreover, the invention can be used to determine the presence or absence of a GBS infection within about 5 minutes of a sample being taken. Thus, the presence or absence of a GBS infection may be determined within about 5 minutes, within about 10 minutes, within about 15 minutes or within about 20 minutes of the sample being taken. Complete analysis can, therefore, be performed at the bedside of a subject/patient. Consequently, the claimed invention is a significant improvement over known methods used to detect a GBS infection (e.g. enrichment culture), which can take a day, two days or even longer. The invention, therefore, provides a shorter time between testing and diagnosis, and thus relieves the burden of EOGBS disease.

If it is determined using a method according to the invention that a subject does not have a GBS infection, they may not require treatment. However, if it is determined that a subject does have a GBS infection, then they may require treatment.

Thus, according to a third aspect, there is provided a method of treating a subject that has a GBS infection, the method comprising:
diagnosing a subject that has a Group B Streptococcus infection using the method according to the first or second aspect, and
administering a therapeutic agent to the subject, in order to treat the infection.

According to a fourth aspect, there is provided a method of treating a subject that has a GBS infection, the method comprising:
administering a therapeutic agent to a subject, who has been diagnosed with a GBS infection using the method according to the first or second aspect, in order to treat the infection.

According to a fifth aspect, there is provided a method of determining if a therapeutic agent is effectively treating a GBS infection in a subject, the method comprising:
determining the concentration of a GBS-VOC in a test sample that has been taken from the genital mucosa of the subject, and
comparing the concentration of the GBS-VOC in the test sample to the concentration in a reference sample,
wherein if the concentration of a GBS-VOC in the test sample is lower compared to the concentration in a reference sample, it may be indicative that the therapeutic agent is effectively treating the Group B Streptococcus infection in the subject.

The reference sample may have been taken from the same subject or a different subject. Preferably, the reference sample is a sample that has been taken from the same subject but at an earlier time point than the test sample. Preferably the earlier sample indicated that the subject was colonized with GBS.

It will be appreciated that the concentration of a GBS-VOC in a test sample positively correlates with the magnitude/severity of a GBS infection. Thus, for example, a reduction in concentration of a GBS-VOC in the test sample compared to the concentration in a reference sample may be indicative of a reduction in the magnitude/severity of the GBS infection. Alternatively, an increase in concentration of a GBS-VOC in the test sample compared to the concentration in a reference sample may be indicative of an increase in the magnitude/severity of the GBS infection.

The concentration of the GBS-VOC in the test sample may be lower by (or reduced by at) least about 5%, 10%, 15%, 20%, 25%, 30%, 35%, 40%, 45%, 50%, 55%, 60%, 65%, 70%, 75%, 80%, 85%, 90%, 95% or 100% compared to the concentration in the reference sample.

According to a sixth aspect, there is provided use of a GBS-VOC as a biomarker for determining if a subject has, or does not have, a GBS infection.

The presence or absence of a GBS infection can be determined based on the presence or absence of a GBS-VOC in the liquid phase and/or the vapour phase (headspace) of the sample. Advantageously, only a small concentration and volume of the liquid phase component is required to determine the presence or absence of a GBS infection. This may be achieved by taking a swab of fluid lining the genital mucosa. Preferably, the method according to the invention is performed on the vapour phase (head space) of the GBS-VOCs of a (liquid) sample. The concentration of a GBS-VOC in the liquid phase of a sample can be calculated based on the concentration of the GBS-VOC in the gaseous phase (headspace) of the sample. Advantageously, only a small volume of the liquid phase component is required to produce a vapour phase that can be used to determine the presence or absence of a GBS infection. Advantageously, the liquid phase of a GBS-VOC can be frozen and thawed without losing its ability to act as a diagnostic biomarker. Thus, GBS-VOCs are robust biomarkers.

It is preferred that a GBS-VOC is selected from the group comprising or consisting of butyraldehyde (butanal), ethyl acetate, methyl acetate and 2-methylpropanal. Thus, the method according to the invention may comprise determining the presence, or absence, of one or more GBS-VOCs selected from the group comprising or consisting of butyraldehyde (butanal), ethyl acetate, methyl acetate and 2-methylpropanal.

It will be appreciated that the detection of a single GBS-VOC may be used to reliably diagnose a subject with a GBS infection. However, the detection of two or more GBS-VOCs may provide a more robust diagnosis. Thus, the method according to the invention may comprise detecting one, two, three or four GBS-VOCs. Preferably, the invention comprises determining the presence of two or more GBS-VOCs.

It will also be appreciated that the absence of a single GBS-VOC may be used to confirm that a subject does not have a GBS infection. However, determining the absence of two or more GBS-VOCs may provide a more robust diagnosis. Thus, the method according to the invention may comprise determining the absence of one, two, three or four GBS-VOCs. Preferably, the invention comprises determining the absence of two or more GBS-VOCs. In addition, the method according to the invention may be complemented by other methods.

The method according to the invention may be performed without culturing or amplifying bacteria within a sample. The method according to the invention may be performed in vitro.

It will be appreciated that the absence or presence and/or concentration of a GBS-VOC may be determined using any suitable method/technique/technology known in the art, such as gas chromatograph-ion mobility spectrometry (GC-IMS) technology, Gas Chromatograph (GC), Gas Chromatograph-Mass Spectrometry (GCMS), Mass Spectrometry (MS), Ion Mobility Spectrometry (IMS), Differential Mobility Spectrometry (DMS), light absorption Spectrometry, Field Asymmetric Ion Mobility Spectrometry (FAIMS), Electronic Nose, Selective-Ion Flow Tube Mass Spectrometry (SIFT-MS), Protein-transfer-reaction-MS, Optical absorbance/Non-dispersive Infra-red and gas sensors (individual or in an array). Preferably, the step of determining if a GBS-VOC is present in a sample that has been taken from the genital mucosa of the subject comprises detecting the absence or presence and/or concentration of a GBS-VOC in a sample by using, for example, GC-IMS technology.

It will be appreciated that the sample may be analysed immediately after being taken from the subject (i.e. it may be a fresh sample). The sample may be placed in a sealed container, such as a universal or a bijoux. Alternatively, the sample may be frozen and stored. Preferably, the frozen sample is stored in a sealed container, such as a universal or a bijoux. The sample may be frozen and stored at a temperature of about −20° C. or about —80° C. or a temperature between about −20° C. and about −80° C. Preferably, the sample is stored at about −80° C. Thus, the method according to the invention may comprise determining if a GBS-VOC is absent or present in a thawed sample. The sample may be thawed by heating the sample. The sample may be heated to a temperature between about 20° C. and about 85° C., between about 30° C. and about 60° C., between about 35° C. and about 45° C. Preferably, the sample is heated to a temperature of about 40° C. The sample may be agitated prior to detection of GBS-VOCs.

The term ‘treat’ can refer to preventing, eradicating or reducing the severity of a GBS infection. Thus, the therapeutic agent referred to herein may prevent, eradicate or reduce the severity of a GBS infection. The therapeutic agent may be an antibiotic. The antibiotic may be a beta lactam or vancomycin. The beta lactam may be penicillin, cephalosporin or ampicillin. The cephalosporin may be selected from the group consisting of: cephazolin, ceftriaxone and cefotaxime.

The ‘subject’ may be suspected of having a GBS infection. The ‘subject’ may be a vertebrate, mammal or domestic mammal. Hence, the method according to the invention may be used to screen, diagnose or treat any animal, for example, a pig, cat, dog, horse, sheep or cow. Preferably, the subject is a human. Most preferably, the subject is a pregnant female. The method according to the invention may be performed on a subject during the first, second or third trimester. The subject may be a pregnant human, female at about 14-36 weeks of gestation.

The term ‘sample’ or ‘test sample’ refers to a biological specimen taken from the body of a subject. The sample may be an ex vivo sample or an in vitro sample. The sample may not be an in vivo sample. Thus, the method according to the invention may not comprise taking a sample from a subject. The sample may be a (biological) tissue. The tissue may be a solid tissue or cells. The sample may be liquid lining the genital mucosa of the subject. The sample may be the headspace of the liquid lining the genital mucosa of a subject. Thus, the method according to the invention may comprise obtaining a headspace sample from the subject. The skilled person will appreciate that the headspace comprises the air surrounding the urine sample, into which the volatile organic compounds evaporate and/or diffuse (the headspace may be within about 0 to 20 cm, 0 to 15 cm or 0 to 10 cm from the subject). Typically, the headspace comprises, or consists of, the air in a closed container containing the sample. Preferably, however, the sample is a liquid lining the mucosa of the genitals, such as vaginal liquid, preputial mucosal liquid or the mucosal liquid within the tip of the penis. A sample of genital liquid may be taken by performing a swab. Preferably, the swab is taken by rotating the swab on the surface of the genital mucosa. The genital mucosa may be a vaginal mucosa or a mucosa within a penis.

“Confirming that a subject does not have a GBS infection” may refer to recording the name and/or an identifier of the patient so that a third party is aware that the subject does not have a GBS infection.

“Confirming that a subject does have a GBS infection” or “diagnosing a subject that has a GBS infection” may refer to recording the name and/or an identifier of the patient so that a third party is aware that the subject does have a GBS infection.

The term “recording” can refer to fixing or storing in writing or digitally (e.g. typed or dictated).

### FIGURE LEGENDS

For a better understanding of the invention, and to show how embodiments of the same may be carried into effect, reference will now be made, by way of example, to the accompanying Figures, in which:

FIG. 1 is a 3D topographic map generated by GC-IMS analysis of the constituents of a GBS positive swab. The x-axis represents the drift time in the IMS and the y-axis represents the retention time of the same chemical in the GC. The non-blue areas are chemical signals be detected by the instrument;

FIG. 2 is an AUC ROC curve based on the analysis of samples taken from pregnant women that are colonised with GBS versus samples taken from pregnant women that are not colonised with GBS. Samples were analysed using a G.A.S. GC-IMS instrument using the VOCs, butyraldehyde (butanal), ethyl acetate, methyl acetate and 2-methylpropanal;

FIG. 3 is a box and whiskers plot representing the distribution of level of probabilities of assigning VOCs outputs to patients with and without GBS. The closer the probability is to 1, the more certainty the classification model has to define the sample group. The analysis was performed based on the VOCs, butyraldehyde (butanal), ethyl acetate, methyl acetate and 2-methylpropanal. Each of the swabs was confirmed as positive or negative for GBS by simultaneously performing an enrichment culture analysis of the sample. Dots adjacent to the “whiskers” correspond to outliers. The whiskers indicate 1.5 IQR; and

FIG. 4 is an intermediate analysis graph that shows the location of VOCs identified by GC-IMS analysis of (A) a GBS positive sample and (B) a GBS negative sample after removal of background VOCs (i.e. non-GBS VOCs), before ROC and probability analysis.

### EXAMPLES

Study Design

An accuracy study was undertaken at one UK hospital (University Hospitals Coventry & Warwickshire) serving a diverse population. The study protocol was approved by the NHS Research Ethics Committee West Midlands Birmingham South on 14th January 2014 (13/WM/0486) and all participants gave written informed consent. The Group B Strep Support charity was consulted prior to the application for funding regarding a patient perspective about the study. Research was carried out according to The Code of Ethics of the World Medical Association (Declaration of Helsinki).

Participants and Test Methods

Women between 14-36 weeks gestation were consented during their attendance to a high risk antenatal clinic for women at an increased risk of spontaneous preterm birth. A speculum examination was performed as per patient routine care. A vaginal swab (reference standard) for microbiology culture and sensitivity testing using the enriched culture method was taken and placed into a nonnutritive transport medium, and concurrently two cotton swabs were used to obtain index test vaginal samples.

The index test swabs were then placed in universal containers and snap frozen in liquid nitrogen and stored at −80° C. Specimens were obtained by gently rotating the swabs across the mucosa of the vagina. Biomedical scientists independently interpreted the reference swab cultures. Demographic data including age at booking pregnancy, BMI, ethnicity and smoking status were collected about each woman (Table 1). Samples were taken in a consecutive series from all women who consented in the clinic, some women consented to samples being taken during every attendance to the clinic.

Chemical Analyzer

Chemical vapour analysis of the index swabs was undertaken in the BioMedical Sensors Laboratory, University of Warwick. Here a Gas Chromatograph-Ion Mobility Spectrometer (GC-IMS) was used. This instrument was chosen over more traditional gas chromatograph mass spectrometer (GCMS) as the basic sensitivity of the instrument is much higher than GCMS, it can use nitrogen/air as the carrier gas (so no need for expensive carrier gases such as helium), has a lower purchase/test cost than GCMS and has a much smaller form factor, making it applicable for a ward setting.

Analysis was undertaken using GC-IMS instrument manufactured by G.A.S. (GC-IMS is also the product name, Dortmund, Germany), which is based on Gas Chromatograph-Ion Mobility Spectrometery principles (GC-IMS). In use, the samples, formed of a mixture of VOCs that emanate from the vaginal swab, are injected into the GC-IMS. These VOCs are preseparated by the GC column (in this case a 30m column), which takes the complex mix of chemicals and separates them based on their interaction with the long column coated with a retentive layer (made of (5%-phenyl)(1%-vinyl)-methylpolysiloxane)). Thus chemicals are eluted from the column at different times (known as the retention time). The pre-separated chemicals exit the GC and enter a drift tube IMS detector. Here the molecules are ionized using a radioactive source (in this case tritium) and then released into the drift tube in a controlled manner. The ions are then moved along the drift tube using an electric field (400 V/cm). At the same time a buffer gas (in this case nitrogen) is fed in the opposite direction to the ions. The resultant impacts between the ions and the buffer gas reduce the velocity of the ions. Thus ions with different sizes and shapes will have different drift times (i.e. the time taken for the ion to be detected by the Faraday plate detector at the end of the drift tube).

Thus ions achieve different velocities, inversely proportional to their size, mass and charge and then are collected on a Faraday plate, to provide a time-dependent signal corresponding with ion mobility. Thus, the larger the ion, the greater the number of impacts and the slower the ion travels along the tube. The device can measure substances in the low ppb range.

Chemical Testing and Analysis

The G.A.S. GC-IMS instrument was used to test 607 samples from 243 women. Samples were briefly stored at −80° C. before being thawed and transferred to a 20 ml glass vial in batches of 20. The vials were then sealed with a crimp top lid fitted with a PTFE septum. In measurement, the index samples were initially placed in a tray and kept refrigerated at 4° C. to reduce unwanted odour emission and sample degradation, whilst other samples were being tested. Prior to measurement samples were heated to 40° C. for 10 minutes. The sample line for the GC-IMS was inserted into the septa of the vial using a needle and 2 mls of sample were then extracted from the vial by syringe and injected into the analytical platform. The machine settings were as follows: E1: 150 ml/min (for the drift tube IMS), E2: 20 ml/min (for the GC column) and the pump at 25%. The total run time was 10 minutes. The temperatures were set to: T1: 45° C., T2: 80° C., and T3: 70° C.

Statistical Analysis

The GC-IMS data was first extracted using the L.A.V. software (v2.2.1, G.A.S, Germany), which converts the data from its native file format to a text file. This was followed by a pre-processing step to reduce the dimensionality of the data, making the statistical analysis less computationally intensive. A typical GC-IMS output file (of a single sample) contains typically 11 million data points. Though the number of data points is high, the information content is sparse, with the all of the values containing non-background information being located around the centre of the dataset. Thus, we are able to crop the central section of the data and then apply a threshold to make the background values all be zero. These values are selected by visual inspection of the data using the LAV software and results in around a 500-fold reduction in the number of non-zero data points. Once completed, the data was analysed using a 10-fold cross validation approach. In each fold, the data was split into a 90% training set and a 10% test set. Features with discriminatory power were identified form the training set using a rank-sum test and 50 features with the lowest p-value were taken forward for classification. Here, five different classifiers we used, specifically sparse logistic regression, random forest, Gaussian process classifier, support vector machine and neural network (this set is commonly used within our pipeline). Once the training models had been created, they were applied to the same features in the test set. This process is repeated ten times until all the data has a test result. This process provided test probabilities for each sample and from this, statistical values, including sensitivity and specificity were calculated.

### Example 1—Vaginal Swab

243 women were swabbed throughout pregnancy corresponding to 607 sets of swabs. The demographics of these women is illustrated in Table 1. The maternal GBS colonisation rates, as defined by a positive enriched culture from vaginal swabs, was 13.6% (corresponding to 33 women).

TABLE 1
Characteristics of the women taking part in the study,
the booking BMI of six patients is not known.

| Number of women with demographic data | 243 | |
|---|---|---|
| Age at booking (years) Mean (SD) | 31.3 | (4.9) |
| Booking BMI (kg/m²) Mean (SD) | 26.3 | (5.7) |
| Parity | n (%) | |
| 0 | 80 | (32.9) |
| 1 | 89 | (36.6) |
| 2 | 49 | (20.2) |
| 3 | 12 | (4.9) |
| 4 | 5 | (2.1) |
| ≥5 | 5 | (2.1) |
| Unknown | 3 | (1.2) |
| Ethnic group | n (%) | |
| White | 192 | (79.0) |
| Mixed | 1 | (0.4) |
| Asian | 23 | (9.5) |
| Black | 22 | (9.1) |
| Other | 5 | (2.1) |

### Example 2—Chemical Identification

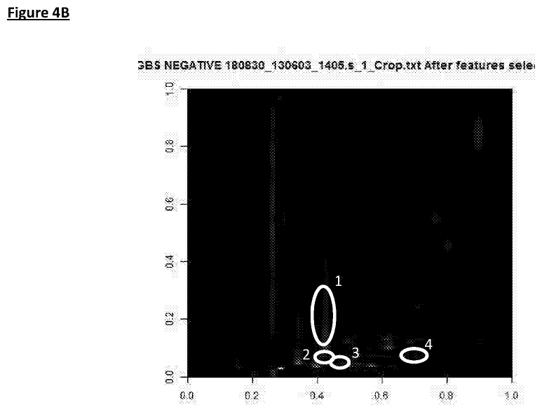

VOC analysis indicates that the presence of butyraldehyde (butanal), ethyl acetate, methyl acetate and 2-methylpropanal can be used to distinguish between samples from subjects that are GBS positive and GBS negative (see FIG. 4A and 4B).

### Example 3—Statistical Analysis

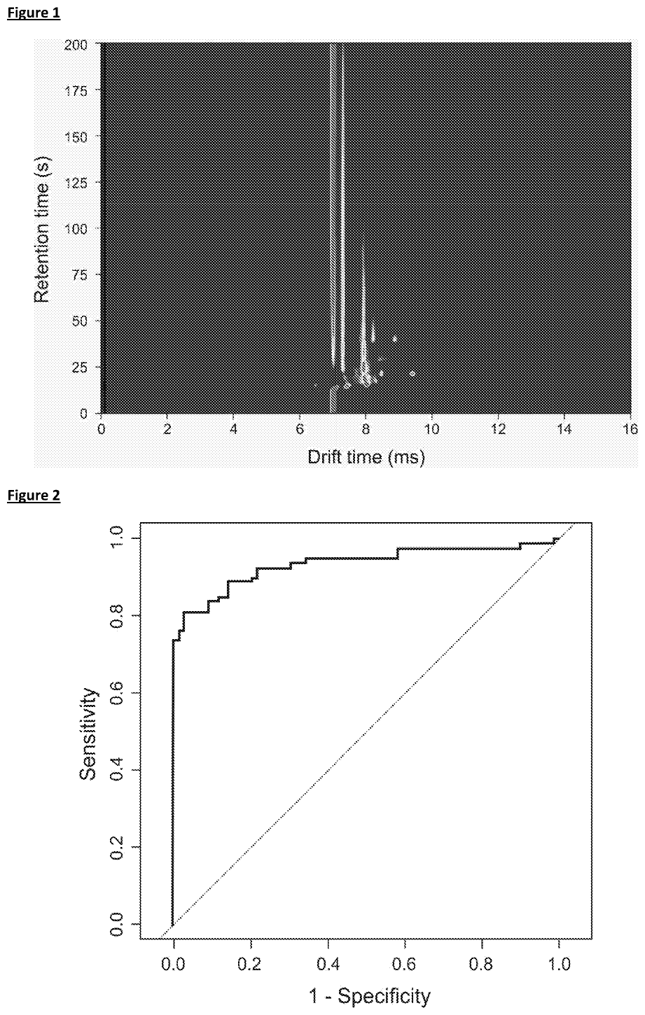

FIG. 1 shows a typical output of the GC-IMS to a positive swab. The output is a 3D topographic map with each point characterised by the retention time in the chromatographic column (in seconds), the drift time in the drift tube (in milliseconds) and the intensity of the ion current signal (in millivolts). The signal intensity is indicated by colour. Each high-intensity area represents a single or combination of chemicals (with the same properties). The long line red line is the RIP (reactive ion peak), which is a background signal. The background is represented in blue with the non-blue areas showing that the instrument is detecting chemicals. The intensity of the peak (with red being the highest intensity) represents the amount of ions (and thus the chemical) detected. In general, each of the circular areas of higher intensity represent a different chemical. Furthermore, it can be see that the majority of the response is in the central section of the output.

The data from the G.A.S. GC-IMS was analysed, as describe above, and the statistical output is shown in Table 2. The results demonstrate a strong VOCs signal (from butyraldehyde (butanal), ethyl acetate, methyl acetate and 2-methylpropanal) is associated with GBS colonisation.

TABLE 2
Statistical output from G.A.S. GC-IMS analytical platform
using a sparse logistic regression classifier.

| Statistical parameter | Value (95% CI) | |
|---|---|---|
| Area under the curve | 0.93 | (0.89-0.98) |
| Sensitivity | 0.81 | (0.71-0.89) |
| Specificity | 0.97 | (0.91-1) |

| | |
|---|---|
| Positive predictive value (PPV) | 0.84 |
| Negative predictive value (NPV) | 0.97 |
| p-value | 2.05E−21 |

An area under the curve value of 0.93 indicates that the VOCs, butyraldehyde (butanal), ethyl acetate, methyl acetate and 2-methylpropanal, are extremely good at discerning between subjects that are GBS positive and GBS negative. This is also supported by a small p-value of 2.05²¹. A sensitivity of 0.81 indicates that there is a probability of 0.81 that a test result will be positive when the subject is GBS positive. A specificity of 0.97 indicates that there is a probability of 0.97 that a test result will be negative when the subject is GBS negative.

A PPV of 0.84 indicates that there is a 0.84 probability that the subject is GBS positive when the test is positive. An NPV of 0.97 indicates that there is a probability of 0.97 that the subject is GBS negative when the test is negative. These results particularly indicate that the VOCs, butyraldehyde (butanal), ethyl acetate, methyl acetate and 2-methylpropanal, are good at identifying subjects that are not GBS positive (in accordance with the first aspect of the invention).

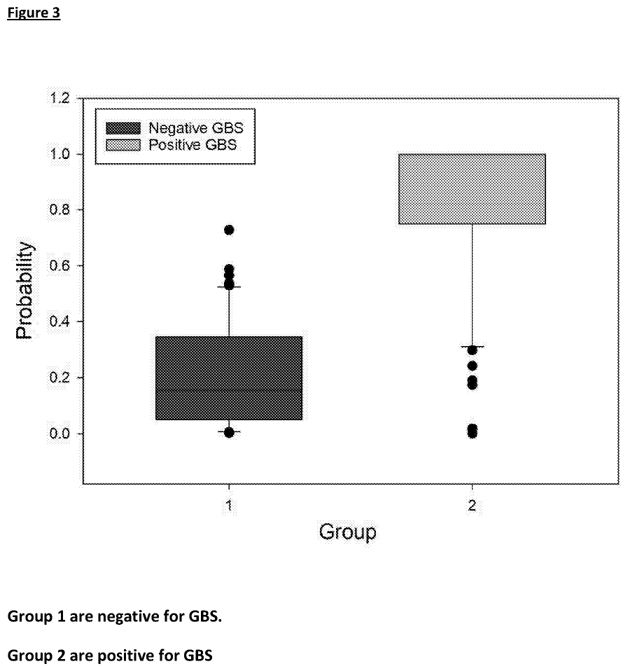

An AUC receiver operator characteristic (ROC) curve is shown in FIG. 2.

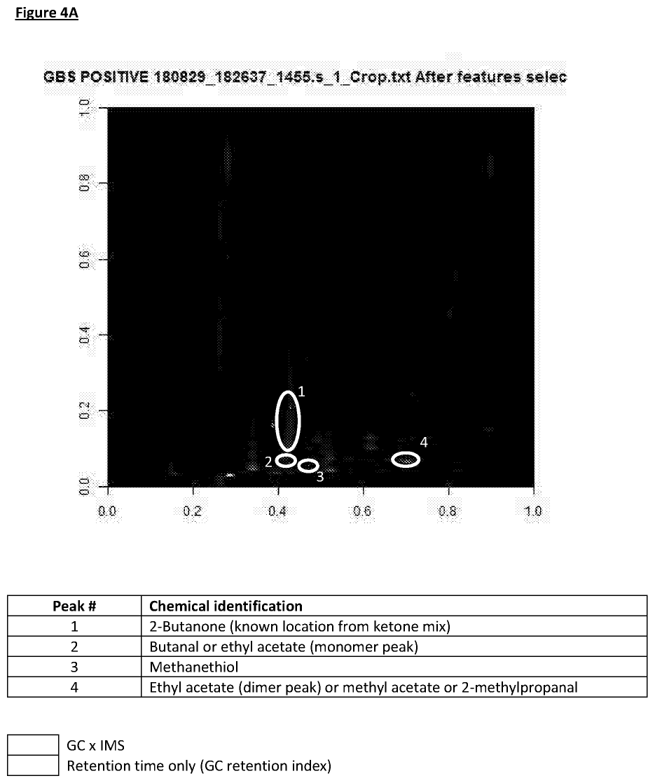

FIG. 3 shows a box and whisper plot with the predicted probability that a sample is positive for GBS. The samples in Group 1 correspond to those taken from pregnant subjects that are confirmed as positive for GBS (by enrichment culture), and the samples in Group 2 correspond to those taken from pregnant subjects that are confirmed as negative for GBS (by enrichment culture). The small degree of overlap between the interquartile range (IQR) of the Group 1 and Group 2 indicates (visually) that butyraldehyde (butanal), ethyl acetate, methyl acetate and 2-methylpropanal,are good biomarkers for ensuring that a sample/subject is correctly identified as GBS positive or GBS negative.

Discussion

Main Findings

The results presented here demonstrate the diagnostic value of vaginal VOCs in the detection of colonisation with GBS. Such a test may be used as a point of care test for women intrapartum, reducing the incidence of EOGBS disease by appropriate administration of antibiotic prophylaxis.

VOC profiles from samples taken from pregnant women colonised with GBS could be discriminated from those who were not colonised with GBS with a high sensitivity and specificity. The results indicate that women who are colonised with GBS have chemically different vaginal swabs to those who are not colonised. The vagina has its own varied microbiome, but the data suggests that despite this, there are differences in VOCs from high vaginal swabs in those who are colonised with GBS. These GBS associated differences in VOCs were demonstrated and are detectable with this novel technology. The inventors believe, although they do not wish to be bound by the theory that the detected VOCs are gaseous waste products that occur as a result of the complex interactions in the vagina between the vaginal and cervical epithelial cells, the vagina flora and the invading GBS pathogens. The data presented shows that GBS colonisation produces a unique VOC fingerprint.

The results presented here demonstrate that the G.A.S. GC-IMS instrument has a very high specificity and negative predictive value for the detection of GBS colonisation. This technology can now be developed as a bedside test for GBS. In the acute intrapartum scenario, women could have a swab taken and analysed in a hand held device in minutes. Where the results are positive, this could guide clinicians to prompt and appropriate administration of intrapartum antibiotics, reducing the risk of EOGBS. The high negative predictive value of the test could be used to counsel families about the low likelihood of colonisation with GBS and bring into question whether administration of antibiotic prophylaxis is necessary, reducing unnecessary antibiotic exposure to both the mother and infant. Furthermore, a large number of women need to be tested for a screening program and the cost of this test is minimal.

Strengths of the results include the large number (n=607) of swabs analysed using the G.A.S. GC-IMS instrument and compared to the reference standard. STARD statement was complied with bias was minimised as far as possible. However, there were a few limitations. The prevalence of colonisation with GBS was lower than expected at 13.6%, compared with the global average of 17.9% and European average 19.0%. In the clinic, women have a vaginal swab only (as part of their screening for risk of spontaneous preterm birth), this is not in keeping with recommendations for specimen collection for detection of colonisation of GBS. Swabbing both the lower vagina and rectum increases the culture yield when compared to sampling the vagina only. Previous studies sub-analysis has illustrated that colonisation from low vaginal swabs only had a mean prevalence of 14.2%, this has more similarity to the cohort used in the Examples.

In conclusion, EOGBS disease remains the leading infectious cause of morbidity and mortality amongst neonates. Preventative efforts have reduced the burden of this disease over time but at present worldwide no universal screening tool or pathway can be agreed. This study has shown that the VOC signature present in vaginal swabs of pregnant women distinguished those swabs from which GBS was detected. Using the G.A.S. GC-IMS analytical platform with a sensitivity and specificity for GBS colonisation of 0.81 and 0.97 respectively. Development of this technology has the potential to provide clinically useful and cost-effective universal screening intrapartum for colonisation with GBS.

1. A screening method for confirming that a subject does not have a Group B Streptococcus (GBS) infection, the method comprising:
determining if a GBS-volatile organic compound (VOCs) is not present in a sample that has been taken from the genital mucosa of the subject,
wherein if a GBS-VOC is not present in the sample, the subject does not have a Group B Streptococcus infection.

2. A method according to claim 1, wherein if a GBS VOC is present in the sample, the subject has a GBS infection.

3. A method of diagnosing that a subject has a Group B Streptococcus (GBS) infection, the method comprising:
determining if a GBS-volatile organic compound (VOCs) is present in a sample that has been taken from the genital mucosa of the subject,
wherein if a GBS-VOC is present in the sample, the subject has a Group B Streptococcus infection.

4. A method of treating a subject that has a GBS infection, the method comprising:
diagnosing a subject that has a Group B Streptococcus infection using the method according to any one of claims 1 to 3, and
administering a therapeutic agent to the subject, in order to treat the infection.

5. A method of treating a subject that has a GBS infection, the method comprising:
administering a therapeutic agent to a subject, who has been diagnosed with a GBS infection using the method according to any one of claims 1 to 3, in order to treat the infection.

6. A method of determining if a therapeutic agent is effectively treating a GBS infection in a subject, the method comprising:
determining the concentration of a GBS-VOC in a test sample that has been taken from the genital mucosa of the subject, and
comparing the concentration of the GBS-VOC in the test sample to the concentration in a reference sample,
wherein if the concentration of a GBS-VOC in the test sample is lower compared to the concentration in a reference sample, it may be indicative that the therapeutic agent is effectively treating the Group B Streptococcus infection in the subject.

7. The method according to claim 6, wherein the reference sample has been taken from the same subject or a different subject.

8. The method according to claim 6 or 7, wherein reference sample is a sample that has been taken from the same subject but at an earlier time point than the test sample.

9. The method according to claim 8, wherein the reference sample taken at the earlier sample indicated that the subject was colonized with GBS.

10. The method according to any one of claims 6 to 9, wherein the concentration of the GBS-VOC in the test sample is lower by (or reduced by) at least about 5%, 10%, 15%, 20%, 25%, 30%, 35%, 40%, 45%, 50%, 55%, 60%, 65%, 70%, 75%, 80%, 85%, 90%, 95% or 100% compared to the concentration in the reference sample.

11. The method according to any one of the preceding claims, wherein the sample is liquid lining the genital mucosa of a subject, such as vaginal liquid.

12. Use of a GBS-VOC as a biomarker for determining if a subject has, or does not have, a GBS infection.

13. The method according to any one of claims 1 to 11 or the use according to claim 12, wherein a GBS-VOC is selected from the group consisting of butyraldehyde (butanal), ethyl acetate, methyl acetate and 2-methylpropanal.
